(19) United States
(12) Patent Application Publication
(10) Pub. No.: US 2014/0011230 A1
(43) Pub. Date: Jan. 9, 2014

(54) Method for Identifying and Screening Dendritic Killer Cells

(71) Applicant: FullHope Biomedical Co., Ltd., Taipei City (TW)

(72) Inventor: Jan-Mou LEE, Taipei City (TW)

(21) Appl. No.: 13/918,529

(22) Filed: Jun. 14, 2013

(30) Foreign Application Priority Data
Jul. 5, 2012 (TW) 101124290

(51) Int. Cl. G01N 33/50

(52) U.S. Cl. CPC G01N 33/5094
USPC 435/34; 435/372; 435/29

(57) ABSTRACT

A method for identifying and screening dendritic killer cells (DKC) is disclosed in the present invention, and the method is to identify a plurality of cell surface markers selected from a group consisting of HLA-G⁻, CD14⁻, CD3⁻, CD19⁻, CD56^{dim} and HLA-DR⁺. The method for screening DKC from human peripheral blood mononuclear cells (PBMC) comprises the following steps. A first cell population with regular cell size is provided and a second cell population is then sorted out from the first cell population by screening the phenotype of CD14 and HLA-G therein. A third cell group is further sorted out from the second cell population by screening the phenotype of CD19 and CD3 therein. Finally, DKC will be identified out of the third cell population by screening the phenotype of CD56 and HLA-DR.

### CROSS-REFERENCE TO RELATED APPLICATIONS

This Non-provisional application claims priority under 35 U.S.C. §119(a) on Patent Application No(s). 101124290 filed in Taiwan, Republic of China, 07, 05, 2012, the entire contents of which are hereby incorporated by reference.

### FIELD OF THE INVENTION

The present invention relates to a method for identifying and screening dendritic killer cells, especially relates to a method for screening out dendritic killer cells with the surface markers of HLA-G⁻CD14⁻CD19⁻CD3⁻CD56^{dim}HLA-DR⁺ from human peripheral blood mononuclear cells by identifying different surface markers of different cells.

### BACKGROUND OF THE INVENTION

Human body will recognize the extraneous matter and start a series of defending process. This defense system is named as immune system. There are many different cells such as leukocytes and lymphocyte, and different protein factors such as immunoglobulins and cytokines working coordinately to protect the body. The immune systems are traditionally divided into innate and adaptive immune systems. Innate immune system is including soluble complement system, polymorphonuclear neutrophils, macrophages and natural killer cells. Adaptive immune system is including humoral and cellular immunity. Humoral immunity as well as cellular immunity involves lymphocyte, lymphokine and immunological memory system. The long-lasting immune memory mounts quick and strong immune responses towards the same pathogen which has invaded the body.

Immune system may respond to different pathogens due to the diversity of major histocompatibility complex (MHC) molecules. The endogenous and exogenous antigens derived from pathogens, are assembled with MHC molecules on the surface of antigen-presenting cells (APC) and then presented to T cells expressing corresponding T cell receptors. MHC in the human beings can be called Human Leukocyte Antigen, HLA, which can be categorized into class I, class II, and class III. HLA class I is widely expressed on all the somatic cells but Class II distribution is restricted to macrophages. B cells and dendritic cells.

Dendritic cells (DC), which have the broadest range of antigen presentation, are professional APC, and named by the appearance of dendrites extending from the cell body. DCs reside in the periphery of body as immature DCs (imDCs). Once pathogen invades human bodies, imDCs capture pathogen-derived antigens, migrate to draining lymph nodes to become mature DCs (mDCs), and present antigens to corresponding T cells there. Therefore, dendritic cells are the starter of the pathogen-specific cellular immune responses.

Natural killer (NK) cells, a key player of innate immune system, spontaneously kill tumor or virally infected cells prior to activation. Mechanisms underlying cytotoxicity of NK cells are grouped into two parts: a) interaction of cell surface tumor necrosis factor superfamily members and their receptors which leads to apoptosis of target cells, (b) release of soluble perform and granzymes. NK cells are rich with small granules in their cytoplasm contain special proteins such as perform and proteases known as granzymes. Upon release in close proximity to a cell slated for killing, perform forms pores in the cell membrane of the target cell through which the granzymes and associated molecules can diffuse in, leading to destruction of target cells. Once virally infected cells or tumor cells have been killed, viral genomic content (CpG or poly I:C), cellular metabolites, and bystander cytokines such as IFN-γ, IL-12 and TNF-α would further activate and augment NK cell activity in term of cytotoxicity and effector cytokine production. Therefore NK cells serve as key innate effector cells targeting to virally infected cells and tumor cells in a non-antigen specific manner while DCs in adaptive immune system trigger antigen-specific cytotoxic T cells which can further clear the infection. Patients deficient in NK cells are proved to be highly susceptible to early phases of herpes virus infection.

Interferon-producing killer dendritic cells (IKDCs), a recently identified leukocyte population in mice, express phenotypes of non-T (CD3⁻), non-B (CD19⁻), intermediate levels of CD11c, and high levels of B220 and NK-specific markers, including NK1.1, DX5, NKG2D and Ly49 family receptors. IKDCs functionally resemble NK cells in cytotoxicity against tumor cells and in production of abundant IFN-γ. On the other hand, upon stimulation with CpG or tumor cells. IKDCs down-regulate NKG2D, up-regulate MHC II, and acquire moderate APC-like activity that activates antigen-specific T cells. Despite acquisition of APC activity after certain stimulations, IKDCs appear to belong to the NK lineage rather than DC lineage. IKDCs express NK-specific Ncr-1 transcripts (encoding NKp46) but not PU.1 that is predominantly expressed in DCs and plasmacytoid DCs. Furthermore, IKDC development parallels NK cells in their strict dependence on the IL-15 cytokine system. Therefore, the putative IKDCs are functionally and developmentally similar to NK cells. Although debates regarding tumoricidal activity and cell lineage development of IKDC were raised herein, further investigations were limited by rare abundance of IKDC in periphery. The frequency of IKDCs in a mouse spleen is below 0.01%, and is even lower in the lymph nodes. Therefore, cumbersome procedure is required for the purification of IKDCs, and the yield is low, This problem has limited the use of IKDCs in research and in application.

### SUMMARY OF THE INVENTION

According to the abovementioned disadvantages of the prior art, Applicant put a lot of efforts in the past years and successfully screens out the cells which have the functions of both cytotoxicity and antigen presenting activity, and defined as dendritic killer cell (hereafter called DKC). That is, the cells have the functions of both natural killer cells and dendritic cells, also be called cytotoxic dendritic cell (cytoDC). Furthermore, the present invention doesn't need to obtain DKC from spleen, lymph node or bone marrow through many complex steps. That is, the present invention provides methods for identifying and screening the DKC from human peripheral blood. Seriously though, the DKC constitutes less than 0.01% in peripheral lymphocytes. Therefore, it is difficult to identify and screen a trace of the DKC from the human peripheral blood.

Therefore, the present invention provides a method for identifying the surface markers of the DKC to successfully screen out the DKC from the human peripheral blood. Once the DKC are successfully sorted out from the human peripheral blood, the research of the DKC can be gone deep into a new aspect such as culture and therapy. It is expected to develop cell immunotherapy of cancer.

The present invention provides a method for identifying DKC involves identifying a plurality of cell surface markers selected from a group consisting of natural killer cell surface markers, dendritic cell surface markers and HLA-G⁻. Preferably, the natural killer cell surface markers comprise CD56⁺. Preferably, CD56⁺ is CD56^{dim}. Preferably, the dendritic cell surface markers comprise HLA-DR⁺. Furthermore, the abovementioned group further consists of a lack of expression of a mononuclear cell surface marker, a B cell surface marker and a T cell surface marker. Preferably, the lack of expression of the mononuclear cell surface marker, the B cell surface marker and the T cell surface marker are CD14⁻, CD19⁻ and CD3⁻, separately. Preferably, the method disclosed in the present invention is applied for identifying the DKC from a human peripheral blood sample. Preferably, the human peripheral blood is obtained from a cancer patient.

The present invention further provides a method for screening DKC, comprising at least following steps. First, a first cell population is provided. And then, a second cell population is sorted out from the first cell population by screening the phenotype of CD14 and HLA-G therein. A third cell population is then sorted out from the second cell population by screening the phenotype of CD19 and CD3 therein. Finally, a dendritic killer cell population is sorted out from the third cell population by screening the phenotype of HLA-DR and CD56 therein.

Preferably, the second cell population comprises the cells with the surface markers of CD14⁻ and HLA-G⁻.

Preferably, the third cell population comprises the cells with the surface markers of CD14⁻, HLA-G⁻, CD3⁻ and CD19⁻.

Preferably, the surface marker of the dendritic killer cell population is CD14⁻HLA-G⁻CD3⁻CD19⁻HLA-DR⁺CD56^{dim}.

Preferably, the method further comprises following steps. First, human peripheral blood is collected and the first cell population is sorted out from the first cell population by the size and the granularity of the cells. Preferably, the human peripheral blood is collected from a cancer patient. Preferably, the first cell population comprises Lymphoid cells and Monocytic cells.

Preferably, the abovementioned sorting steps are performed by using a flow cytometer.

The present invention further provides an isolated human dendritic killer cell, wherein the DKC is HLA-G⁻CD19⁻CD3⁻CD56⁺HLA-DR⁺

The features and advantages of the present invention will be understood and illustrated in the following specification and FIGS. 1-4.

### BRIEF DESCRIPTION OF THE DRAWINGS

FIG. 1 is diagram showing the flow chart of a method for screening dendritic killer

FIG. 2A to FIG. 2D are diagrams showing the result of using a flow cytometer to sort out dendritic killer cells from human peripheral blood of a cancer patient in the preferred embodiment of the present invention;

FIG. 3A to FIG. 3D are diagrams showing the result of using a flow cytometer to sort out dendritic killer cells from human peripheral blood of a healthy donor in the preferred embodiment of the present invention;

FIG. 4 is diagram showing the percentage of dendritic killer cells in human peripheral blood of the cancer patient and the healthy donor;

FIG. 5 is diagram showing the expression amount of the surface markers which is defined according to the result of the flow cytometer;

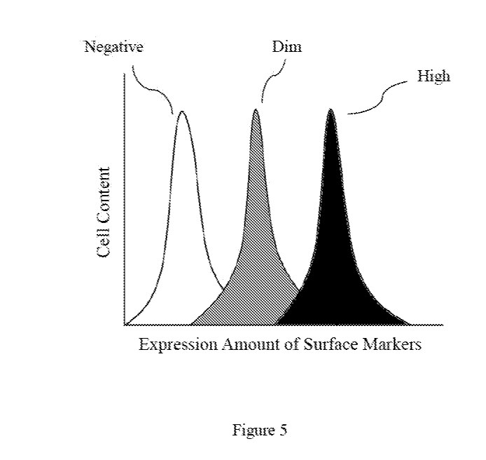

FIG. 6A to FIG. 6B are diagrams showing the efficiency of dendritic killer cells screened by the preferred embodiment of the present invention; and

FIG. 7A to FIG. 7B are diagrams showing the antigen presenting cell activity of dendritic killer cells screened by the preferred embodiment of the present invention.

### DETAILED DESCRIPTION OF THE INVENTION

While the making and using of various embodiments of the present invention are discussed in detail below⁻, it should be appreciated that the present invention provides many applicable inventive concepts that can be embodied in a wide variety of specific contexts. The specific embodiments discussed herein are merely illustrative of specific ways to make and use the invention and do not delimit the scope of the invention. To facilitate the understanding of this invention, a number of terms are defined below. Terms defined herein have meanings as commonly understood by a person having ordinary skill in the art relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “dendritic killer cells” or “DKC” is intended to refer to the cells with both cytotoxicity and antigen presenting cell (APC) activity.

As used herein, the term “HLA-G” is intended to refer to a cell surface marker presented on the surface of regulatory cells, and the regulatory cells usually secrete suppressive cytokines.

As used herein, the term “natural killer cell surface marker” is intended to refer to a cell surface marker presented on the surface of natural killer cells, such as CD56, CD16 or Kp46.

As used herein, the term “dendritic cell surface marker” is intended to refer to a cell surface marker presented on the surface of dendritic cells, such as HLA-DR, CD11c and CD123.

As used herein, the term “mononuclear cell surface marker” is intended to refer to a cell surface marker presented on the surface of mononuclear cells, such as CD14.

As used herein, the term “B cell surface marker” is intended to refer to a cell surface marker presented on the surface of B cell, such as CD19.

As used herein, the term “'F cell surface marker” is intended to refer to a cell surface marker presented on the surface of T cell, such as CD3.

As used herein, the symbol “+” means that the cell surface marker expresses on the surface of the cells and has a larger expressed amount measured by flow cytometer than that of the negative control.

As used herein, the symbol “−” means that the cell surface marker does not express on the surface of the cells and has an expressed amount equal to that of the negative control.

As used herein, the term “dim” means that the cell surface marker expresses on the surface of the cells but has a smaller expressed amounts. That is, the term “dim” is defined as a cell population with a few but larger expressed amounts than that of the negative control.

Preferably, all abovementioned expressed amount of the cell surface markers are measured by flow cytometer, however, the present invention is not limited thereto.

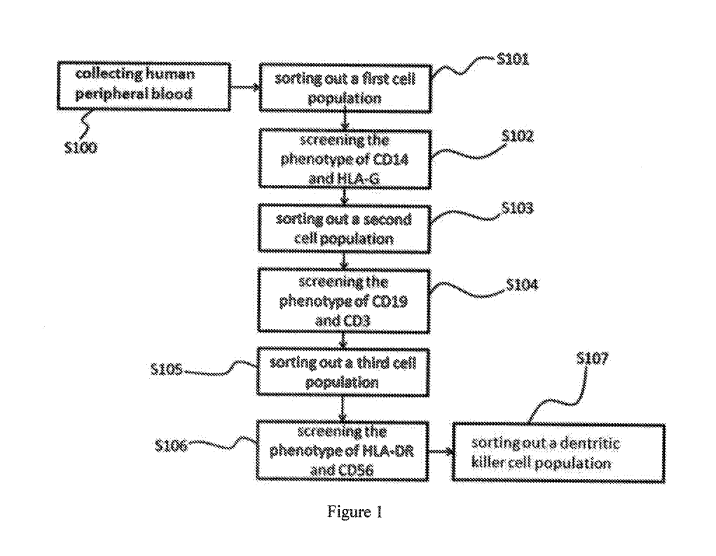

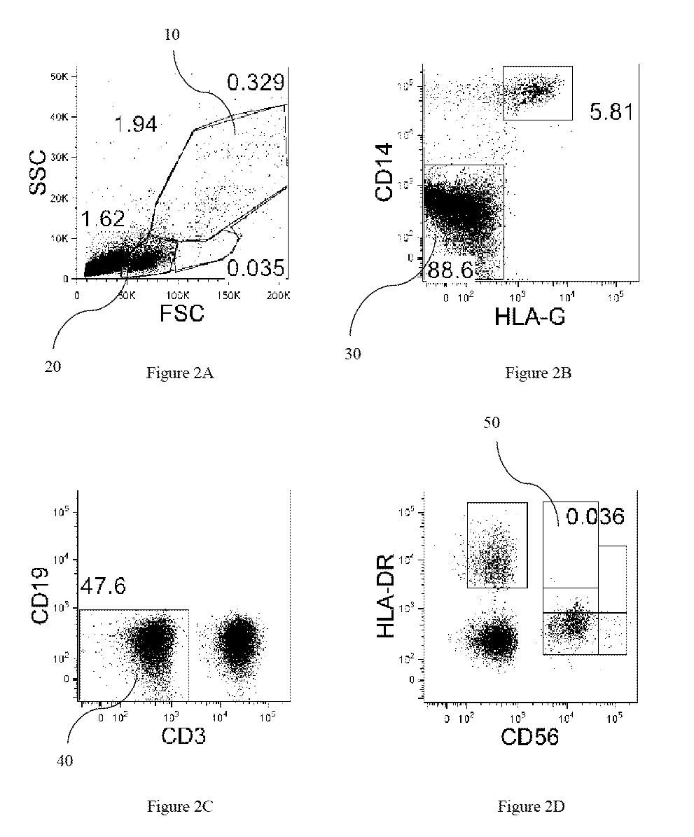

Please refer to FIG. 1 and FIG. 2A to FIG. 2D. FIG I is diagram showing the flow chart of a method for screening DKC in an preferred embodiment of the present invention, and FIG. 2A to FIG. 2D are diagrams showing the result of using a flow cytometer to sort out DKC from human peripheral blood of a cancer patient in the preferred embodiment of the present invention.

First, human peripheral blood mononuclear cells will be separated from 40 mL of the whole blood, which is collected from a cancer patient or a healthy donor respectively, in step S100. It is noted that the whole blood will be diluted by phosphate buffered saline solution with the same volume and centrifuged by Ficoll 1.077 to obtain the human peripheral blood mononuclear cells. The human peripheral blood mononuclear cells comprise the following five categories of cells: monocytic cells, small cells, lymphoid cells, large cells and large and granular cells. Therefore, the distribution of each category of the abovementioned cells will be shown in FIG. 2A by utilizing the flow cytometer in the present invention. Please refer to FIG. 2A, a vertical axis represents the size of the cells, and a transverse axis represents the granularity of the cells. In Step S101, monocytic cells 10 as shown in the central right of FIG. 2A and lymphoid cells 20 as shown in the lower left corner of FIG. 2A are screened out as a first cell population by utilizing the flow cytometer. Preferably, the human peripheral blood is collected

The disclosed methods are amenable to the use of various types of equipment, including one or more sample preparation robots and one or more flow cytometers to identify or screen the cell population by cell surface markers. Flow cytometry allows for single cell analysis at speeds far surpassing any other single cell analysis technology in the art. This enables a statistically significant number of cells to be analyzed faster than using other alternative techniques.

There are many different types of sample preparation robots and liquid handlers that are known in the art. In one embodiment, a flow cytometer is used with any suitable sample preparation robot or liquid handler that is known in the art. Furthermore, a single laser flow cytometer is used in an embodiment for the analyzing step. In another embodiment, a multi-laser flow cytometer is used for the analyzing step and the present invention is not limited thereto.

The development and optimization of an extensive set of fluorochromes and conjugating chemistries allows for a variety of ligands, such as immunoglobulins and small molecules, to be conjugated to the fluorochromes. Lasers with emission lines ranging from the ultraviolet to the red region of the light spectrum can excite these fluorochromes. Consequently, a large number of spectrally distinct reagents can be used to label cells for study with fluorescence-based instrumentation such as flow cytometry used in the present invention. These reagents are well known in the art. In one embodiment, one or more fluorochromes are used during the analyzing step. In some embodiments, one or more stains are used in the analysis of cellular responses to drug compositions.

Please continue referring to FIG. 1; the step S102 of screening the phenotype of CD14 and HLA-G of the first cell population is performed by the flow cytometer. And then, the cells as shown in the lower left corner of FIG. 2B with the surface markers of CD14⁻ and HLA-G⁻ are sorted out as a second cell population 30 in the step S103.

The step S104 of screening the phenotype of CD19 and CD3 of the second cell population is performed. Please refer to FIG. 2C, a third cell population 40 with the surface markers of CD3⁻ and CD19⁻ is then sorted out from the second cell population 30 in the step S105. It is noted that CD3 is the surface marker of T cell, and CD19 is the surface marker of B cell. That is, the step S105 is performed to remove T cell and B cell from the second cell population 30.

The phenotype of HLA-DR and CD56 of the third cell population 40 is screened in the step S106. And then, the cells distributed at the upper right corner of FIG. 2D will be sorted out in the step S107. That is, the sorted cells are the cells with the surface markers of HLA-DR⁺ and CD56^{dim}, and the sorted cells are DKC disclosed in the present invention.

According to the abovementioned steps S100·S107, the present invention provides a method for identifying DKC and involves identifying a plurality of cell surface markers selected from a group consisting of HLA-G⁻, CD56⁺ and HLA-DR⁺. Preferably, the abovementioned group further consists of CD14⁻, CD19⁻and CD3⁻. Preferably, CD56⁺ is CD56^{dim}. That is to say, the DKC with the surface markers of HLA-G⁻CD14⁻CD19⁻CD3⁻CD56^{dim}HLA-DR⁺ can be successfully screened out from the human peripheral blood by combining the abovementioned surface markers with the steps S100˜S107.

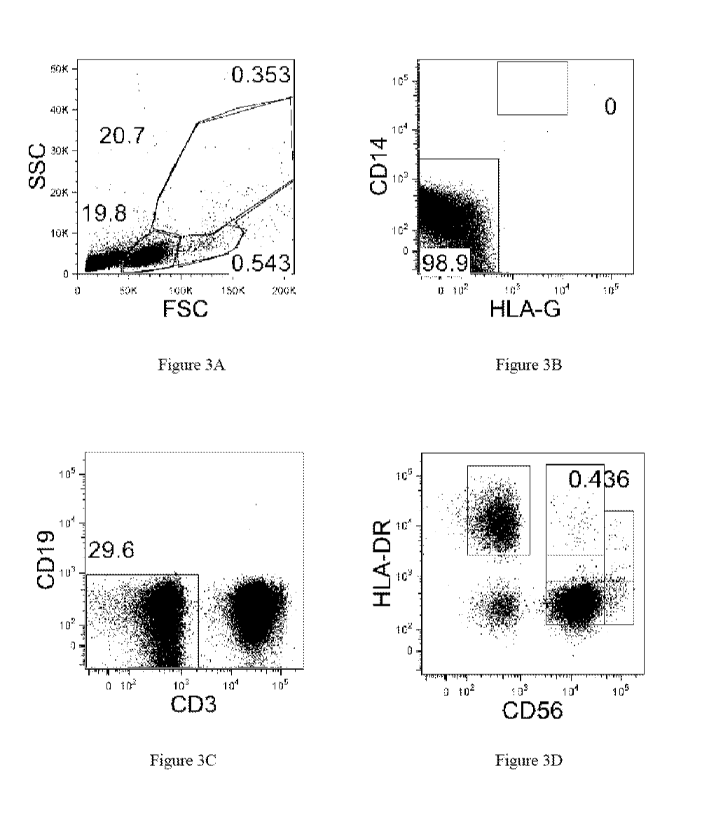

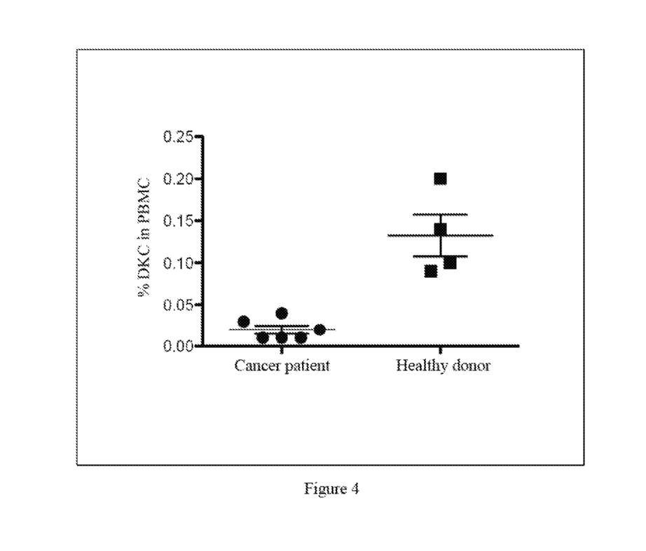

Please refer to FIG. 3A to FIG. 3D; FIG. 3A to FIG. 3D are diagrams showing the result of using a flow cytometer to sort out DKC from human peripheral blood of a healthy donor in the preferred embodiment of the present invention. The method disclosed in FIG. 3A to FIG. 3D is basically the same as that disclosed in FIG. 2A to FIG. 2D. The only difference is the source of the human peripheral blood. The human peripheral blood used in FIG. 3A to FIG. 3D is collected from a healthy donor. The method described from FIG. 3A to FIG. 3D is to sort out the monocytic cells and the lymphoid cells according its size and granularity. And then, the phenotype of HLA-G⁻, CD14⁻, CD3⁻, CD19⁻, CD56⁺ and HLA-DR⁺ will be detected in order to sort out the DKC with the surface marker of HLA-G⁻CD14⁻CD19⁻CD3⁻CD56^{dim}HLA-DR⁺. Please refer to the upper right corner of FIG. 3D, the content of the DKC existed in the human peripheral blood of the healthy donor is actually higher than that of the cancer patient. Please also refer to FIG. 4 which is diagram showing the percentage of the DKC in human peripheral blood of the cancer patient and the healthy donor, it is noted that the percentage of the DKC in the human peripheral blood of the cancer patient is around 14.5% of that in the healthy donor.

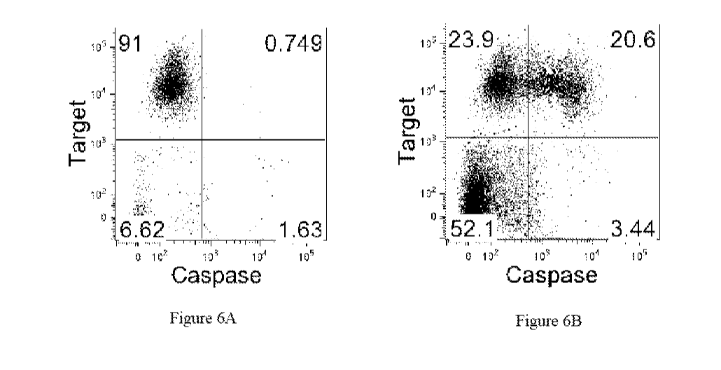

In order to prove the efficiency of natural killer cells which the DKC has, Applicant reacts the sorted DKC with target tumor cells and further utilizes the flow cytometer to detect the apoptosis of the target cells. As shown in FIG. 6A and FIG. 6B, the vertical axis represents the sizes of the cells and the transverse axis represents the content of Caspase 6 in the cells. It is noted that Caspase 6 is an important protease in apoptosis so that the cell is dead or dying if Caspase 6 of the cell is dyed. That is, the killing efficiency of the DKC can be detected by detecting the content of Caspase 6 in the target cells. Please continue referring to FIG. 6A and FIG. 6B, each of the figures is divided into four blocks: the upper portion represents the target cells, the lower portion represents the DKC, the left portion represents the living cells and the right portion represents the dead cells. Preferably, the target cells are K562 cells,

Please refer to FIG. 6A, there is no dyed Caspase 6 existed in the target cells before reacting the target cells with the DKC, and all the target cells distribute at the upper left portion of FIG. 6A. After reacting the target cells with the DKC, the cells distributed at the upper portion of FIG. 6A apparently shift to right as shown in FIG. 6B. That is, a lot of the target cells decease after reacting with the DKC. On the other hand, the DKC distributed at the lower portion of FIG. 6B are alive after reacting with the target cells. Therefore, the killing efficiency of the DKC identified and screened by the method disclosed in the present invention is actually proved through the abovementioned results.

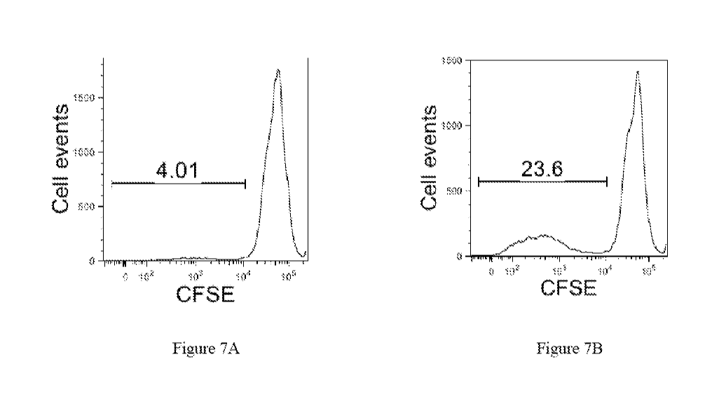

In order to prove the antigen presenting cell activity of the DKC, Applicant culture the DKC generated from PBMC of the first subject with PBMC of the second subject. And then, the T cell activation and proliferation in PBMC of the second subject will be measured by flow cytometer. Please refer to FIG. 7A and FIG. 7B, a mixed leukocyte reaction is utilized to label the peripheral blood mononuclear cells sorted from the peripheral blood of the second subject with CFSE. And then, the peripheral blood mononuclear cells labeled with CFSE will be reacted with the DKC of the first subject, and T cell of the second subject will be activated. Therefore, the activation and proliferation of T cell of the second subject can represent allo-stimulatory APC of DKC generated from PBMC of the first subject. The abovementioned CFSE is a dye for quantifying the degree of cell proliferation. After each cell proliferation, the fluorescence intensity of CFSE will decrease. However, when CFSE labeling is performed optimally, approximately 7-8 cell divisions can be identified before the CFSE fluorescence is too low to be distinguished above the autofluorescence background. Thus CFSE represents an extremely valuable fluorescent dye for immunological studies, allowing lymphocyte proliferation, migration and positioning to be simultaneously monitored.

Please refer to FIG. 7B; it shows a result of reacting the DKC and T cell labeled with CFSE. As shown in the figures, the vertical axis represents the content of the cells and the transverse axis represents the fluorescence intensity of CFSE within the cells. Please continue referring to FIG. 7B, 23.6% T cell of the second subject have been activated by the DKC to processing cell proliferation after reacting the DKC of the first subject with T cell of the second subject labeled with CFSE. That is, the DKC identified and screened by the present invention actually has the antigen presenting cell activity. Please refer to FIG. 7A; FIG. 7A is negative control. In negative control, there are only the T cells of the second subject in medium, and only 4.01% of T cell has been activated and proliferate.

To sum up, the present invention provides a method for identifying the surface markers of the DKC to successfully screen out the DKC from the human peripheral blood. Once the DKC are successfully sorted out from the human peripheral blood, the research of the DKC can be gone deep into a new aspect such as culture and therapy. It is expected to develop cell immunotherapy of cancer.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims

What is claimed is:

1. A method for identifying dendritic killer cell (DKC) involving identifying a plurality of cell surface markers selected from a group consisting of natural killer cell surface markers, dendritic cell surface markers and HLA-G⁻.

2. The method according to claim 1, wherein the natural killer cell surface markers comprise CD56⁺.

3. The method according to claim 2, wherein CD56⁺ is CD56^{dim}.

4. The method according to claim 1, wherein the dendritic cell surface markers comprise HLA-DR⁺.

5. The method according to claim 1, wherein the group further consists of a lack of expression of a mononuclear cell surface marker, a B cell surface marker and a T cell surface marker.

6. The method according to claim 5, wherein the lack of expression of the mononuclear cell surface marker, the B cell surface marker and the T cell surface marker are CD14⁻, CD19⁻and CD3⁻.

7. The method according to claim 1, the method can identify the dendritic killer cell from a human peripheral blood sample.

8. The method according to claim 7, wherein the human peripheral blood is obtained from a cancer patient.

9. A method for screening dendritic killer cell, comprising at least following steps:
providing a first cell population;
sorting out a second cell population from the first cell population by screening the phenotype of CD14 and HLA-G therein;
sorting out a third cell population from the second cell population by screening the phenotype of CD19 and CD3 therein; and
sorting out a dendritic killer cell population from the third cell population by screening the phenotype of HLA-DR and CD56 therein.

10. The method according to claim 9, wherein the second cell population comprises the cells with the surface markers of CD14⁻ and HLA-G.

11. The method according to claim 9, wherein the third cell population comprises the cells with the surface markers of CD14⁻, CD3⁻and CD19⁻.

12. The method according to claim 9, wherein the surface marker of the dendritic killer cell population is CD14⁻HLA-G⁻CD3⁻CD19⁻HLA-DR⁺CD56^{dim}.

13. The method according to claim 9, further comprising following steps:
collecting human peripheral blood; and
sorting out a first cell population by the size and the granularity of the cells.

14. The method according to claim 13, wherein the human peripheral blood is collected from a cancer patient.

15. The method according to claim 9, wherein the first cell population comprises Lymphoid cells and Monocytic cells.

16. The method according to claim 9, wherein the sorting steps are performed by using a flow cytometer.

17. An isolated human dendritic killer cell, wherein the DKC is HLA-G⁻ CD14⁻ CD19⁻CD3⁻ CD56⁺ HLA-DR⁺.
